(19) United States
(12) Patent Application Publication
(10) Pub. No.: US 2019/0077721 A1
(43) Pub. Date: Mar. 14, 2019

(54) METHOD OF MANUFACTURING ORGANIC FERTILIZERS BY USING ORGANIC RAW MATERIAL, ANTAGONISTIC MICROORGANISM, FERMENTATIVE MICROORGANISM, AND SYNTHETIC MICROORGANISM, AND ORGANIC FERTILIZERS MANUFACTURED BY SAID MANUFACTURING METHOD

(71) Applicant: Yoon Soo CHOI, Seoul (KR)

(72) Inventor: Yoon Soo CHOI, Seoul (KR)

(21) Appl. No.: 15/765,592

(22) PCT Filed: Oct. 18, 2015
(86) PCT No.: PCT/KR2016/011667

§ 371 Date: Apr. 3, 2018

(30) Foreign Application Priority Data
Oct. 21, 2015 (KR) 10-2015-0146915

(51) Int. Cl. C05F 11/08; C12N 9/88; C12N 9/00

(52) U.S. Cl. CPC C05F 11/08; C12N 9/88; A01N 63/02; C12Y 600/00; C12Y 401/00; C12N 9/93

(57) ABSTRACT

The present invention relates to a method for manufacturing an organic fertilizer prepared using antagonistic microorganisms, fermentative microorganisms, synthetic microorganisms, and organic raw materials, and to an organic fertilizer manufactured by the manufacturing method. The present invention has the technical feature wherein a powder-type organic raw material characterized by being produced via a contaminant removal step, a sterilization step, a microorganism culture medium preparation step, and a microorganism additive solution preparation step are subjected to a mixing step, a fermentation step, and a drying step. The present invention is effective for soil improvement, crop growth, and disease and pest control. In addition, fertilizer produced through the technical solution contains primary metabolites, secondary metabolites, conjugated enzymes, antibiotics, bioactive substances and inducers, etc., and thus not only does not contaminate the soil, but is also effective for ecosystem restoration, and for increasing profits through the production of safe organic crops.

### TECHNICAL FIELD

The present invention relates to an organic fertilizer using antagonistic, fermentative and synthetic microorganisms and a method for manufacturing an organic fertilizer, and more particularly, to a method for manufacturing an organic fertilizer using antagonistic, fermentative and synthetic microorganisms prepared through single, mixed or combined cultivation of antagonistic, fermentative and synthetic microorganisms isolated from soil for soil improvement, crop growth and disease and insect pest control and an organic raw material, and an organic fertilizer produced by the method.

### BACKGROUND ART

In modern agriculture, generally, due to continuous use and overuse of chemical fertilizers, pesticides and herbicides, soil, water and crops are polluted, and humans as well as ecosystems are destroyed.

Particularly, soil contamination is directly related to soil microorganisms, so that the number of microorganisms is reduced, even more and more species become extinct, and soils gradually become desertified.

While farmers who realize the seriousness of ecological destruction have applied various types of traditional agricultural techniques to restore their ecosystems, the restoration of ecosystems is a difficult problem unless organic materials are used in agriculture without agricultural pesticides and chemical fertilizers, or herbicides, which are the main causes of ecological destruction.

Today, lime and silicates are used as a soil modifier in Korea. However, when lime and silicates are continuously used, they are accumulated, thereby hardening the soil and causing various disorders in crops.

In addition, although microbial agricultural materials among national certified organic materials have been put to practical use, due to a lack of awareness of microorganisms, the microbial agricultural materials are misused, and exhibited insignificant effects.

Meanwhile, since effective microorganism (EM) materials, which were invented in Japan and have been used in various countries around the world, can have an effect only if microorganisms survive environmental influences such as temperature, moisture, pH, a soil type of CEC or ORP, and competition with indigenous microorganisms, and useful materials produced while the survival microorganisms settle and proliferate in soil and crops are provided to the soil and the crops, are used in accordance with complicated and difficult conditions for use, including a method for use, the frequency of use, a soil condition, a weather condition, a cultivation area, storage according to a farming environment, and soil microbiota is improved when continuously sprayed for a long time, it is difficult for common farmers to use such EM materials.

As related prior art, there are Korean Unexamined Patent Application Publication No. 10-2010-0032855 (Title of the Invention: Microbial Agent for Promoting Plant Growth and Method Using the Same, Publication Date: Mar. 26, 2010) and Korean Unexamined Patent Application Publication No. 10-2010-0130842 (Title of the Invention: Method for Increasing Antimicrobial Activity of Lysosome Isolated from Eukaryotes and Eco-Friendly Antibiotic Obtained Using the Same, Publication Date: Dec. 14, 2010).

### DISCLOSURE

### Technical Problem

The present invention, which was designed to solve the above-mentioned problems of the conventional art, is directed to providing an organic fertilizers using antagonistic, fermentative and synthetic microorganisms and a method for manufacturing an organic fertilizer, and particularly, a method for manufacturing an organic fertilizer prepared using antagonistic, fermentative and synthetic microorganisms and an organic raw material according to the present invention, by culturing the antagonistic, fermentative and synthetic microorganisms isolated from soil through single, mixed or combined cultivation for soil improvement, crop growth and disease and insect pest control, and an organic fertilizer produced by the method.

Other objects of the present invention can be construed by the features of the present invention, further clarified by exemplary embodiments of the present invention, and realized by means and combinations shown in the scope of the present invention.

### Technical Solution

To achieve the problems solved by the present invention, the present invention has technical features as follows.

A method for manufacturing an organic fertilizer using antagonistic, fermentative and synthetic microorganisms and an organic raw material according to the present invention includes a mixing step for producing a mixture by spraying a microorganism additive solution produced by a step of culturing microorganisms by any one or more of single, mixed or combined cultivation of antagonistic, fermentative and synthetic microorganisms isolated from soil, a step of preparing a microorganism culture medium by adding any one or more of complex amino acids to primary metabolites, secondary metabolites, complex enzymes such as lyases and ligases, antibiotics, bioactive substances and inducers, which are generated in the step of culturing microorganisms, and a step of preparing a microorganism additive solution for mixing the microorganism culture medium prepared in the step of preparing a microorganism culture medium with a molasses diluent; onto a powder-type organic raw material, which is produced by a foreign material removal step for removing impurities and foreign materials of an organic raw material, a sterilization step for sterilizing the organic raw material with steam, and a crushing step for crushing the organic raw material; a fermentation step for fermenting the mixture produced in the mixing step; and a drying step for drying the mixture having undergone the fermentation step.

In the method for manufacturing an organic fertilizer using antagonistic, fermentative and synthetic microorganisms and an organic raw material according to the present invention, the step of culturing microorganisms includes a first cultivation step for single or mixed cultivation of any one or more of the antagonistic, fermentative and synthetic microorganisms and a second culture step for combined cultivation of the microorganisms having undergone the first cultivation step.

In the method for manufacturing an organic fertilizer using antagonistic, fermentative and synthetic microorganisms and an organic raw material according to the present invention, the first cultivation step is performed by any one or more of commensalism, synergism and mutualism between the microorganisms.

In the method for manufacturing an organic fertilizer using antagonistic, fermentative and synthetic microorganisms and an organic raw material according to the present invention, the second culture step is for producing any one or more of primary metabolites, secondary metabolites, complex enzymes such as lyases and ligases, antibiotics, bioactive substances and inducers by exchanging genes through interspecies communication using a signal substance between populations, such as a microbial pheromone.

In the method for manufacturing an organic fertilizer using antagonistic, fermentative and synthetic microorganisms and an organic raw material according to the present invention, the step of culturing microorganisms is for producing functional microorganisms exhibiting any one or more functions of soil improvement, crop growth, disease and insect pest control, biodegradation of organic contaminants, and purification of metal contaminants through gene exchange among the introduced microorganisms.

In the method for manufacturing an organic fertilizer using antagonistic, fermentative and synthetic microorganisms and an organic raw material according to the present invention, the step of preparing microorganisms is for culturing the microorganisms up to each of a lag phase, an exponential phase and a stationary phase.

In the method for manufacturing an organic fertilizer using antagonistic, fermentative and synthetic microorganisms and an organic raw material according to the present invention, the step of culturing microorganism consists of a 9 step transfer procedure by culture tank, and is for culturing the microorganisms by sequentially culturing the microorganisms in various media in Step 1, an Erlenmeyer flask in Step 2, a 20 L bioreactor in Step 3, a 0.5 ton culture tank in Step 4, 1 ton culture tank in Step 5, 2.5 ton culture tank in Step 6, 5 ton culture tank in Step 7, 10 ton culture tank in Step 8, and a bulk culture tank in Step 9.

In the method for manufacturing an organic fertilizer using antagonistic, fermentative and synthetic microorganisms and an organic raw material according to the present invention, the organic raw material in the step of removing foreign materials is prepared by mixing 74 wt % of cereal bran, 10 wt % of expeller cake, 10 wt % of roasted chaff and rice straw flour, and 6 wt % of an additive.

In the method for manufacturing an organic fertilizer using antagonistic, fermentative and synthetic microorganisms and an organic raw material according to the present invention, the additive is prepared by mixing any one or more of grinded fish meal, grinded bone meal and grinded crab shell meal.

In the method for manufacturing an organic fertilizer using antagonistic, fermentative and synthetic microorganisms and an organic raw material according to the present invention, the molasses diluent in the step of preparing a microorganism additive solution is prepared by mixing 20 to 40 wt % of molasses with 60 to 80 wt % of purified water.

In the method for manufacturing an organic fertilizer using antagonistic, fermentative and synthetic microorganisms and an organic raw material according to the present invention, the step of preparing a microorganism additive solution is for mixing 25 to 50 wt % of the molasses diluent with 50 to 75 wt % of the microorganism culture medium.

In the method for manufacturing an organic fertilizer using antagonistic, fermentative and synthetic microorganisms and an organic raw material according to the present invention, the mixing step is for mixing the mixture prepared in the previous step to have a moisture content of 50 to 60%.

In the method for manufacturing an organic fertilizer using antagonistic, fermentative and synthetic microorganisms and an organic raw material according to the present invention, the fermentation step starts with stirring of the mixture at the time when a fermentation temperature reaches 40° C. to 45° C. and the fermentation temperature is maintained at 50° C. or less.

In the method for manufacturing an organic fertilizer using antagonistic, fermentative and synthetic microorganisms and an organic raw material according to the present invention, the fermentation step is for performing fermentation for 3 to 10 days.

In the method for manufacturing an organic fertilizer using antagonistic, fermentative and synthetic microorganisms and an organic raw material according to the present invention, the fermentation step consists of a first fermentation step in which a microorganism additive solution prepared with fermentative microorganisms and synthetic microorganisms is sprayed onto the organic raw material to allow fermentation for 3 to 5 days and a second fermentation step in which a microorganism additive solution prepared with antagonistic microorganisms is sprayed to allow fermentation for 3 to 5 days.

The organic fertilizer manufactured using antagonistic, fermentative and synthetic microorganisms and an organic raw material according to the present invention is manufactured by any one of the methods shown in Claims 1 to 15.

In the method for manufacturing an organic fertilizer using antagonistic, fermentative and synthetic microorganisms and an organic raw material according to the present invention, the organic fertilizer continuously imparts useful substances to soil and crops by means of metabolisms of the antagonistic, fermentative and synthetic microorganisms.

In the method for manufacturing an organic fertilizer using antagonistic, fermentative and synthetic microorganisms and an organic raw material according to the present invention, the organic fertilizer provides habitats and a substrate by loading the cultured antagonistic, fermentative and synthetic microorganisms into the organic raw material, thereby protecting growth of the microorganisms.

### Advantageous Effects

As described above, the present invention provides an organic fertilizer manufactured using antagonistic, fermentative and synthetic microorganisms, and a method of manufacturing the organic fertilizer, and therefore can have effects of soil improvement, crop growth and disease and insect pest control.

In addition, the fertilizer produced according to the present invention contains primary metabolites, secondary metabolites, complex enzymes such as lyases and ligases, antibiotics, bioactive substances and inducers, and thus cannot contaminate soil, and can have effects of restoring an ecosystem and producing safe organic products, leading to an increase in income.

The fertilizer of the present invention provides habitats and a substrate by loading microorganisms into an organic raw material, and therefore also has an effect of solving problems of conventional microbial agents, which are survival, settlement and proliferation of microbes.

Further, the microorganisms loaded into the organic fertilizer start to proliferate when conditions such as a soil temperature of 10° C. or more and a saturated moisture content of 20 wt % or more are maintained, thereby continuously releasing useful substances, and therefore microbiota of soil and crops can be improved within a short period of time.

Furthermore, since the microorganisms and useful substances are loaded into the organic raw material and then dried, the fertilizer of the present invention can be easily handled and stored for a long time.

### DESCRIPTION OF DRAWINGS

FIG. 1 is a flow chart illustrating a method for manufacturing an organic fertilizer using antagonistic, fermentative and synthetic microorganisms and an organic raw material according to the present invention.

FIG. 2 is a photograph showing that the organic fertilizer manufactured using antagonistic, fermentative and synthetic microorganisms and an organic raw material according to the present invention is top-dressed for a soil improvement test.

FIG. 3 is a photograph showing that the organic fertilizer manufactured using antagonistic, fermentative and synthetic microorganisms and an organic raw material according to the present invention is top-dressed for a chili pepper growth test.

FIG. 4 is a photograph showing that the organic fertilizer manufactured using antagonistic, fermentative and synthetic microorganisms and an organic raw material according to the present invention is top-dressed for a disease and pest control test.

FIG. 5 is a photograph showing that the organic fertilizer manufactured using antagonistic, fermentative and synthetic microorganisms and an organic raw material according to the present invention is top-dressed for a no-tillage test.

FIG. 6 is a photograph showing that the organic fertilizer manufactured using antagonistic, fermentative and synthetic microorganisms and an organic raw material according to the present invention is top-dressed for a weed control test.

### MODES OF THE INVENTION

A detailed description of the present invention, which will be described below, refers to accompanying drawings illustrating specific exemplary embodiments of the present invention. The exemplary embodiments will be sufficiently described in detail to implement the present invention by those of ordinary skill in the art. It should be understood that various embodiments of the present invention are different, but do not need to be mutually exclusive. For example, particular shapes, structures and characteristics described herein may be embodied differently without departing from the spirit and scope of the present invention with regard to the embodiments. In addition, it should be understood that the position or arrangement of an individual component disclosed in each embodiment may be modified without departing from the spirit and scope of the present invention. Therefore, the following detailed description is not to be taken in a limited sense, and the scope of the present invention is to be limited only by the accompanying claims, along with the full range of equivalents to that claimed by the claims. In the drawings, like reference numerals denote the same or similar functions throughout several aspects.

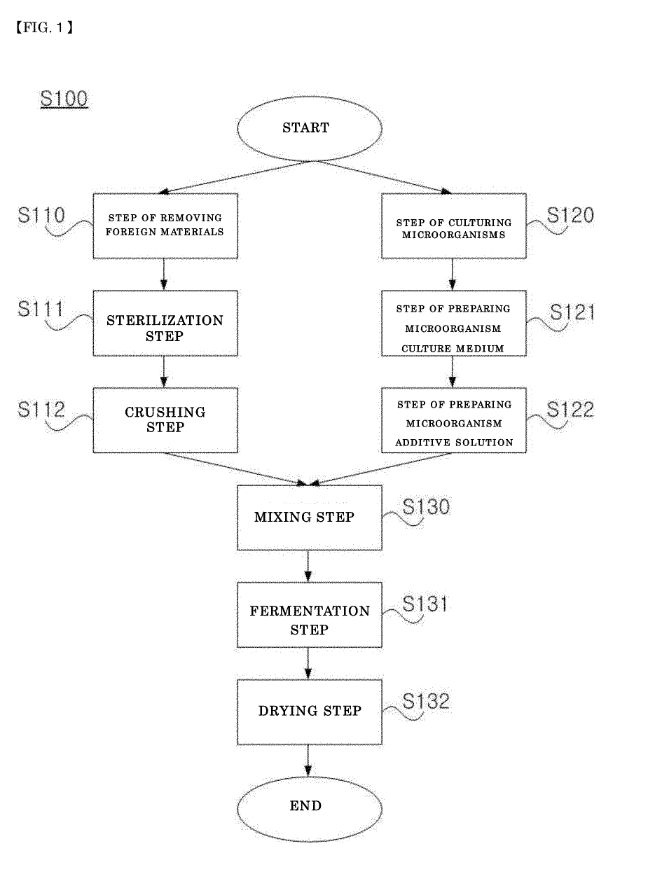

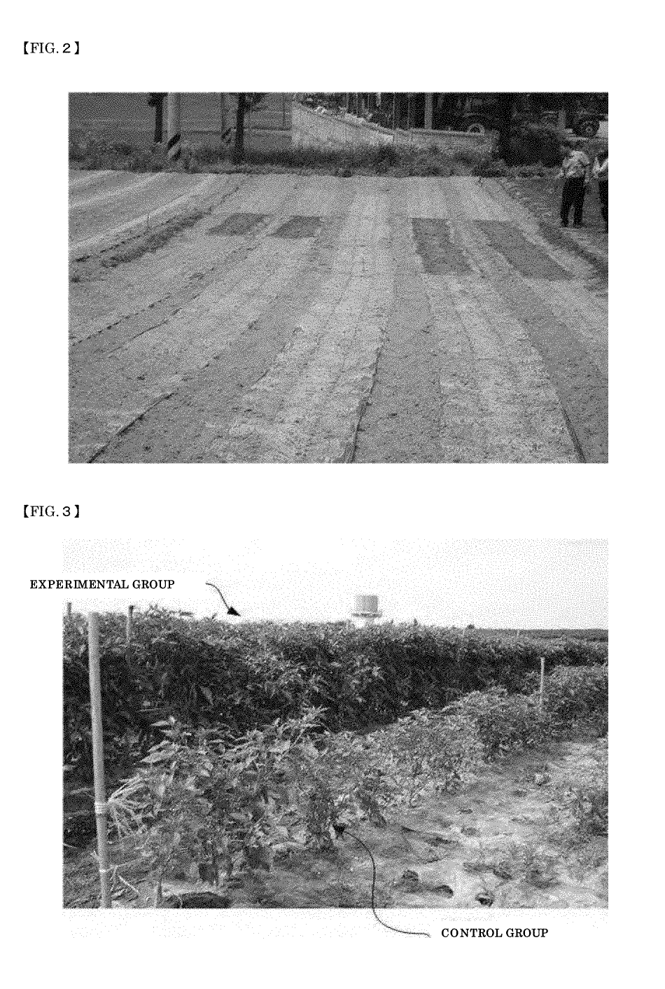

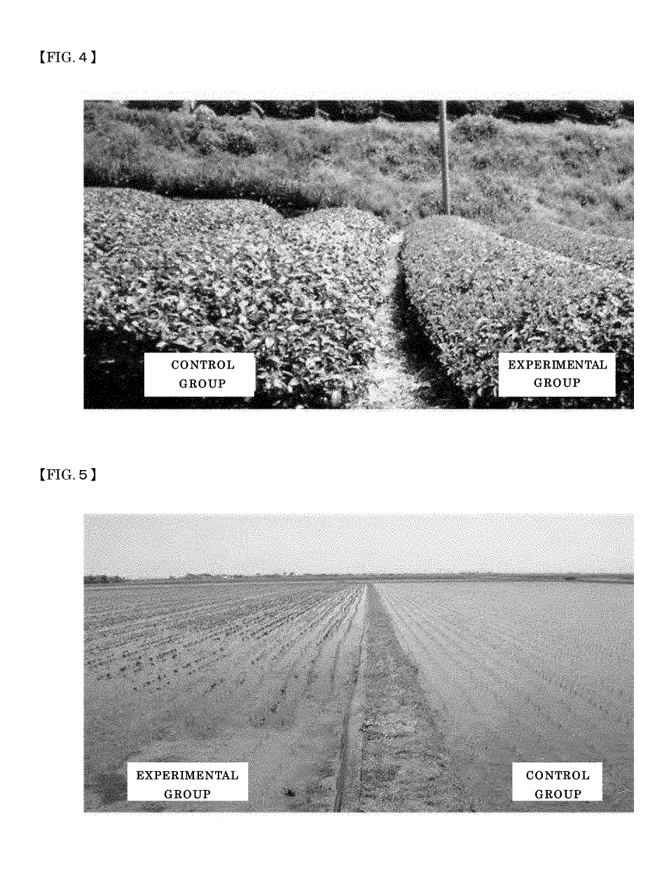

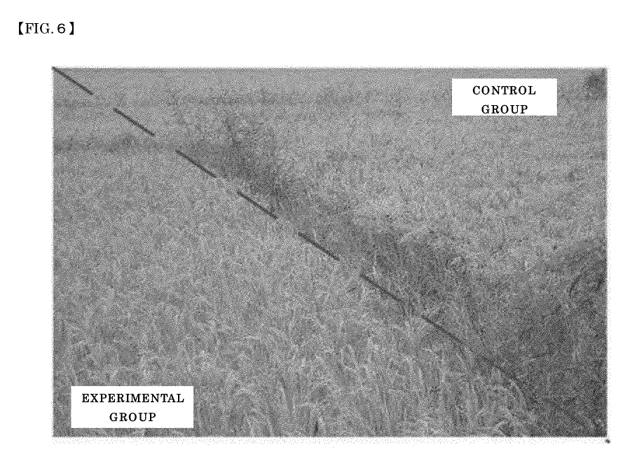

FIG. 1 is a flow chart illustrating a method for manufacturing an organic fertilizer using antagonistic, fermentative and synthetic microorganisms and an organic raw material according to the present invention, FIG. 2 is a photograph showing that the organic fertilizer manufactured using antagonistic, fermentative and synthetic microorganisms and an organic raw material according to the present invention is top-dressed for a soil improvement test, FIG. 3 is a photograph showing that the organic fertilizer manufactured using antagonistic, fermentative and synthetic microorganisms and an organic raw material according to the present invention is top-dressed for a chili pepper growth test, FIG. 4 is a photograph showing that the organic fertilizer manufactured using antagonistic, fermentative and synthetic microorganisms and an organic raw material according to the present invention is top-dressed for a pest control test, FIG. 5 is a photograph showing that the organic fertilizer manufactured using antagonistic, fermentative and synthetic microorganisms and an organic raw material according to the present invention is top-dressed for a no-tillage test, and FIG. 6 is a photograph showing that the organic fertilizer manufactured using antagonistic, fermentative and synthetic microorganisms and an organic raw material according to the present invention is top-dressed for a weed control test.

A method S100 for manufacturing an organic fertilizer using antagonistic, fermentative and synthetic microorganisms and an organic raw material according to the present invention includes a mixing step S130 for spraying and mixing a microorganism additive solution produced by a step of culturing microorganisms S120, a step of preparing a microorganism culture medium S121 and a step of preparing a microorganism additive solution S122 with a powder-type organic raw material produced by a step of removing foreign materials S110, a sterilizing step S111 and a crushing step S112; a fermentation step S131; and a drying step S132.

The powder-type organic raw material is produced by the step of removing foreign materials S110, the sterilizing step S111 and the crushing step S112.

The step of removing foreign materials S110 is for removing impurities and foreign materials from an organic raw material.

The foreign materials are removed by a chemical/physical method according to the type of an additive, for example, grinded fish meal, grinded bone meal and grinded crab shell meal.

The organic raw material in the step of removing foreign materials S110 is a raw material in which 74 wt % of cereal bran, 10 wt % of expeller cake, 10 wt % of roasted chaff and rice straw flour, and 6 wt % of an additive are mixed.

The additive in the step of removing foreign materials S110 contains any one or more of grinded fish meal, grinded bone meal and grinded crab shell meal.

The sterilizing step S111 is for sterilizing the organic raw material with steam.

Steam sterilization is for killing the microorganisms due to irreversible coagulation and denaturation of enzymes and tissue proteins by setting a temperature and a pressure according to a material to be sterilized and spraying saturated water vapor obtained by heating.

In addition, compared to gas sterilization, the steam sterilization is not toxic, has fast penetration into the material to be sterilized, and is less expensive.

The crushing step S112 is for crushing the organic raw material.

The microorganism additive solution may be absorbed within a short period of time by crushing and increasing a surface area of the organic raw material

When the microorganism additive solution is sprayed after crushing the organic raw material, it may be evenly mixed.

The microorganism additive solution is produced through a step of culturing microorganisms S120, a step of preparing a microorganism culture medium S121, and a step of preparing a microorganism additive solution S122.

The step of culturing microorganisms S120 is for culturing antagonistic microorganisms (Bacillus sp., Streptomyces sp., Penicillium sp., or Trichoderma sp.), fermentative microorganisms (Lactobacillus sp., Saccharomyces sp., or Aspergillus sp.) and synthetic microorganisms (Rhodobacter sp., Azotobacter sp. or Pseudomonas sp.), which are isolated from soil, by any one of the methods including single cultivation, mixed cultivation and combined cultivation.

The step of culturing microorganisms S120 includes a first cultivation step S120s for single cultivation or mixed cultivation of any one or more of the antagonistic microorganisms, the fermentative microorganisms and the synthetic microorganisms, and a second culture step S120b for combined cultivation of the microorganisms having undergone the first cultivation step.

The microorganism culture is for blocking the invasion of foreign microbes after target microbes are inoculated into a nutrient-added medium and growing the target microbes.

The single cultivation is for culturing one type of microorganisms, and the mixed cultivation is for culturing a mixture of several types of microorganisms.

Combined cultivation refers to second cultivation of an optimal combination of microorganisms selected from the single-cultured microorganisms and mixed-cultured microorganisms to obtain a target material.

The first cultivation step S120a is performed by any one or more of commensalism, synergism and mutualism between the microorganisms.

Commensalism is a symbiotic relationship in which part A benefits, and part B has neither a benefit nor disadvantage, and part A unilaterally relies on part B, which however is not an absolute relationship. It means that part B has neither an advantage nor disadvantage, but does not have an adverse effect on part A.

Synergism is a symbiotic relationship in which both part A and part B benefit and can independently survive, however, the relationship is not absolute.

Mutualism is an extended concept of synergism, and an absolute symbiotic relationship in which both parts A and B benefit. In this case, part A imparts a benefit to part B, and also benefits, and part A and part B are not replaced with each other.

In the second cultivation step S120b, aerobic, anaerobic or semi-aerobic microorganisms having undergone the first cultivation step produce any one or more of primary metabolites, secondary metabolites, complex enzymes such as lyases and ligases, antibiotics, bioactive substances and inducers by exchanging genes of populations through interspecies communication using a signal substance, a microbial pheromone, between populations in the second cultivation step.

According to the step of culturing microorganisms S120, functional microorganisms exhibiting any one or more functions of soil improvement, crop growth, disease and insect pest control, biodegradation of organic contaminants, and purification of metal contaminants through gene exchange between introduced microorganisms are produced.

In the step of culturing microorganisms S120, the microorganisms are cultured up to a lag phase, an exponential phase and a stationary phase.

The growth cycle of the microorganisms consists of a lag phase which is a period of physiologically adapting microbes exposed to a new environment after inoculation into a new medium, an exponential phase which is a period of exponentially growing microbes, a stationary phase which is a period of constantly maintaining a viable cell count and reaching the maximum total cell count, and a death phase which is a period of inhibiting further growth of microbes due to the depletion of nutrients and a great number of microbes. In this phase, the microbes die or enter a dormant state to survive until being able to use new nutrients, thereby decreasing the viable cell count.

In the step of culturing microorganisms S120, to prevent a decrease in viable cell counts due to a poor growth environment caused by metabolic wastes of the microorganisms, the microorganisms are cultured until the stationary phase, which is followed by the death phase.

The step of culturing microorganisms S120 consists of a 9-step transfer procedure by culture tank, in which the microorganisms are sequentially cultured in the first step performed in various media, the second step performed in an Erlenmeyer flask, the third step performed in a 20 L bioreactor, the fourth step performed in a 0.5 ton culture tank, the fifth step performed in an 1 ton culture tank, the sixth step performed in a 2.5 ton culture tank, the seventh step performed in a 5 ton culture tank, the eighth step performed in a 10 ton culture tank, and the ninth step performed in a bulk culture tank.

In the step of culturing microorganisms S120, the number of microorganisms is increased through the transfer procedure.

The cultured antagonistic, fermentative and synthetic microorganisms are loaded into an organic raw material to provide habitats and a substrate, and to protect the growth of the microorganisms.

When the microorganisms are loaded into an organic fertilizer to provide habitats and a substrate, useful substances produced by metabolisms of the antagonistic, fermentative, synthetic microorganisms loaded into the organic raw material may be continuously provided to soil and crops.

The useful substances provided by the metabolisms of the microorganisms are substances that are essential for crop growth, for example, nitrogen, phosphate, potassium, amino acids, vitamins, proteins, saccharides, lipids, and cellulose.

The antibiotics and inducers produced by the metabolisms of the antagonistic, fermentative, synthetic microorganisms loaded into the organic raw material may inhibit the growth of bacterial pathogens and fungal pathogens in crops or kill the pathogens, and increase sensitivity and resistance of the crops, thereby protecting crop growth. When the soil is top-dressed with the organic fertilizer, and a soil temperature of 10° C. or more and a saturated moisture content of 20% or more are maintained, the antagonistic, fermentative and synthetic microorganisms loaded into the organic fertilizer start to proliferate, and dominate the rhizosphere and the phyllosphere of crops, resulting in the improvement of microbiota of the soil and crops as antagonistic, fermentative and synthetic types.

In the step of preparing a microorganism culture medium S121, a microorganism culture medium is prepared by adding any one or more of complex amino acids to the primary metabolites, secondary metabolites, complex enzymes such as lyases and ligases, antibiotics, bioactive substances and inducers produced in the microorganism cultivation step S120.

In the step of preparing a microorganism culture medium S121, the microorganism culture medium is prepared by adding a different amount of the complex amino acids per group of microorganisms when the complex amino acids are added.

The reason for such difference is that there are microorganisms requiring a large amount of complex amino acids necessary for microbial growth, and microorganisms requiring none or only a small amount of complex amino acids.

In the step of preparing a microorganism additive solution S122, the microorganism culture medium prepared in the step of preparing a microorganism culture medium S121 is mixed with a molasses diluent.

In the step of preparing a microorganism additive solution S122, the molasses diluent is a molasses diluent prepared by mixing 20 to 40 wt % of molasses with 60 to 80 wt % of purified water.

Molasses, which is the main ingredient of the molasses diluent, refers to a liquid from which crystalline sugar is separated when sugar is produced.

The substrates of the microorganisms are mainly organic materials, and the molasses diluent contains a sugar which becomes a substrate of most microorganisms, among the organic materials.

When a concentration of the molasses diluent is too high or low, since it can have an influence on the growth of the microorganisms in the fermentation step S131, a desired fermentation effect may not be observed.

In the step of preparing a microorganism additive solution S122, 25 to 50 wt % of the molasses diluent is mixed with 50 to 75 wt % of the microorganism culture medium.

Depending on a culture environment of microbiota, a microorganism additive solution is prepared by controlling contents of the molasses diluent and the microorganism culture medium.

In the mixing step S130, the powder-type organic raw material and the microorganism additive solution are sprayed and mixed.

In the mixing step S130, the powder-type organic raw material is mixed with the microorganism additive solution to have a moisture content of 50 to 60 wt %.

In the mixing step S130, when the mixture has a moisture content of 50 to 60 wt %, an environment for stably fermenting the microorganisms is created, and in the fermentation step S131, fermentation is effectively accomplished.

In the fermentation step S131, the powder-type organic raw material and the microorganism additive solution, which are mixed in the mixing step S130 are fermented.

In the fermentation step S131, stirring starts from the time when a fermentation temperature of the mixture reaches 40° C. to 45° C., and the fermentation temperature is maintained at 50° C. or less.

In the fermentation step S131, since the mixture is fermented to continuously generate heat, the fermentation temperature is maintained at 50° C. or less by means of a cooling apparatus or natural cooling.

In the fermentation step S131, fermentation is performed for 3 to 10 days.

When the fermentation period in the fermentation step S131 is 3 days or less, the organic raw material is not properly fermented, and therefore a desired fertilizer effect cannot be expected.

In addition, when the fermentation period in the fermentation step S131 is 10 days or more, since the organic raw material is matured, there is almost no active ingredient in the organic fertilizer, and most of the microorganisms die, and therefore a desired fertilizer effect may not be expected.

In the fermentation step S131, fermentation is carried out by a first fermentation step S131a in which a microorganism additive solution prepared using fermentative microorganisms and synthetic microorganisms is sprayed onto the organic raw material to allow fermentation for 3 to 5 days and a second fermentation step S131b in which a microorganism additive solution prepared using antagonistic microorganisms is sprayed to allow fermentation for 3 to 5 days.

In the drying step S132, the mixture having undergone the fermentation step S131 is dried.

In the drying step S132, the mixture was dried by injecting external air into the organic fertilizer fermented in a fermentation tank while stirring.

The organic fertilizer 100 prepared by the method for manufacturing an organic fertilizer using antagonistic, fermentative and synthetic microorganisms and an organic raw material according to the present invention S100.

The antagonistic, fermentative and synthetic microorganisms, which are loaded, in a dormant state, into the organic fertilizer 100 prepared using the antagonistic, fermentative and synthetic microorganisms and the organic raw material start to proliferate when a soil temperature is maintained at 10° C. or more, and a saturated moisture content of 20% or more is provided, and therefore microbiota in the soil and crops are improved as antagonistic, fermentative and synthetic types.

As shown in FIG. 2 illustrating a soil improvement test performed after the organic fertilizer prepared using the antagonistic, fermentative and synthetic microorganisms and the organic raw material was used as a top-dressing, soil with normal soil fertility was improved to antagonistic, fermentative or synthetic soil by top-dressing the organic fertilizer 100 prepared using the antagonistic, fermentative and synthetic microorganisms and the organic raw material, and then chili peppers were cultivated by the same top-dressing method for 5 years. As a result, every year, the chili peppers were strong against disease and pests, a large amount of high quality chili peppers were harvested, physical, chemical and biological properties of the soil were improved annually, and the soil fertility was highly maintained.

In addition, low molecular amino acids and organic acids produced by the antagonistic, fermentative and synthetic microorganisms allow the diffusion of clay particles or ionized minerals of soil to maintain a distance between the molecules at a certain level or more and to make them be in a state in which an inorganic chemical reaction cannot progress (chelated). Therefore, salts are not accumulated, inorganic nutrients required for crops were slowly absorbed, and the inorganic chemical reaction was inhibited, resulting in the formation of soil with high buffering capacity.

In addition, chain polymers produced by the antagonistic, fermentative and synthetic microorganisms have high activity with respect to non-ions, and react with clay particles and inorganic materials, which are in a non-ionic state by being diffused into low molecular amino acids and organic acids, resulting in formation of a soil aggregate.

In addition, the aggregate is increased, thereby forming a water-stable aggregate without destruction due to rain, and therefore, the physical properties of the soil are improved, and the chemical and biological properties are also improved.

In addition, after first to second stages of cultivation, it was confirmed that the soil was improved by approximately pH 1 to 2, a hydroxyl group (OH)-containing inorganic compound, that is, a base, is in a state in which it is difficult to be ionized. Alkali soil is close to neutral and has high ion exchange capacity, thereby temporarily increasing pH to 8.5 or more. In this case, alkalization is caused by digestion of hydrogen ions (H⁺) by photosynthetic bacteria, and although pH is increased or alkalization progresses, there is no stress, and the pH temporarily increased over time is decreased and then stabilized near neutral. However, even when the soil is acidic, pH is slowly increased without using an alkali material such as lime.

In addition, the synthesis capability of the soil was improved as water and air permeabilities of the soil were improved, soil water was always kept clean due to supply of anions and enzymes with high activity to soil, and root activity was increased.

Moreover, the antagonistic, fermentative and synthetic microorganisms may solubilize insoluble nutrients generated by salt accumulation, thereby eliminating salt stress and degrading agricultural pesticide components and environmental hormones.

As shown in FIG. 3 illustrating a chili pepper growth test performed after the organic fertilizer manufactured using the antagonistic, fermentative and synthetic microorganisms and the organic raw material was top-dressed, as a result of cultivating chili peppers in soil top-dressed with the organic fertilizer 100 prepared using the antagonistic, fermentative and synthetic microorganisms and the organic raw material, the chili peppers have slightly small, thick and straight leaves due to photosynthesis and current promotion, and therefore, the light well penetrates into the chili peppers, thereby increasing a photosynthetic activity, and white fine roots expand to several layers, thereby increasing an absorption area, resulting in the harvest of a large amount of high quality chili peppers.

In addition, during the metabolism of crops, organic energy based on carbon and oxygen and utilizing sunlight as an energy source is combined with nitrogen, thereby synthesizing amino acids, and a protein is formed by condensing the amino acids. When crops absorb a large amount of inorganic nitrogen, to convert nitrogen into a protein, saccharides produced by photosynthesis are used to be in equilibrium with the nitrogen, and therefore, there is no capacity to accumulate nutrients. Such capacity means actual quantity and quality. In an organic fertilizer 100-treated group, since most organic materials are solubilized and absorbed into an organic substance such as an amino acid, in the process of protein synthesis, the required ratio of saccharides is low, and even if inorganic nitrogen is absorbed, soluble saccharides are absorbed at the same time, thereby consuming less saccharides synthesized in leaves, and therefore, there is capacity to accumulate saccharides. As a result, the crops of the organic fertilizer 100-treated group have an increased accumulation of saccharides, resulting in the harvest of a large amount of high quality crops.

As shown in FIG. 4 illustrating a pest control test performed after the organic fertilizer manufactured using the antagonistic, fermentative and synthetic microorganisms and the organic raw material was top-dressed, in the pest control test using a pathogen causing pepper anthracnose, performed after top-dressing the organic fertilizer 100 prepared using the antagonistic, fermentative and synthetic microorganisms and the organic raw material, one half of a chili pepper field is set as a control group, the other half is set as a treated group, and then the entire field of chili peppers is inoculated by a pathogen causing pepper anthracnose, such as Colletotrichum coccodes is inoculated into young chili peppers grown up to five leaves in a form of flowing through leaves.

For the control group, irrigation was carried out without other treatments after anthracnose was caused, and for the treated group, after the onset of anthracnose, the organic fertilizer 100 was applied by injected fertilization at 200 g per injection and sufficient irrigation was carried out. As a result of the test, almost all chili peppers in the control group had anthracnose and thus were no longer tested, and in the treated group, only some of the pepper plants had anthracnose and showed a symptom of leaf soft rot, were grown again and taller, and had larger fruits, thereby increasing a yield.

Such a result indicates that antibiotics immobilized in the organic fertilizer 100 and the loaded antagonistic, fermentative and synthetic microorganisms survive in the rhizosphere and phyllosphere of the peppers, the antibiotics are produced and continuously provided to the crops, and anthracnose pathogenicity is lost due to a process of transformation through gene transfer and inducers.

In addition, disease and pests are generated due to perturbation of a metabolic system by inadequate growth environments for crops, such as an increase in temperature and excessive humidity, a lack of oxygen due to an increase in respiration of roots, continuous cropping, root burning due to overuse of fertilizers, inorganic nutrients and harmful substances accumulated in soil, which act as various physiological stresses, the equilibrium of the metabolism is broken, and thus a large amount of unstable harmful reducing materials, for example, undigested nitrogen components such as ammonia, nitric acid and amides, are produced in the crops.

These reducing materials are odorous and act to inhibit cell and enzyme activity, and most disease and pests are attracted by these substances and proliferate by feeding on the substances.

The disease and pests are fundamentally blocked by improving soil in antagonistic, fermentative and synthetic states using the organic fertilizer 100 to facilitate temperature control, humidity control and oxygen supply due to air and water permeabilities of the soil, solving a problem of continuous cropping or a salt stress by using a soil aggregate, and providing the optimal growth environments without giving a stress to the crops.

As shown in FIG. 5 illustrating a no-tillage test performed after the organic fertilizer manufactured using the antagonistic, fermentative and synthetic microorganisms and the organic raw material was top-dressed, as a result of the no-tillage test performed after top-dressing the organic fertilizer 100 prepared using the antagonistic, fermentative and synthetic microorganisms and the organic raw material, soil was improved with the organic fertilizer 100 as an antagonistic, fermentative and synthetic type to also effectively use a live organic material as organic energy, soil water was purified, air and water permeabilities were improved by softening the soil due to a soil aggregate, and a range of root expansion was widened and therefore it was possible to cultivate crops without tillage, which did not cause any problem.

As shown in FIG. 6 illustrating a weed control test performed after the organic fertilizer manufactured using the antagonistic, fermentative and synthetic microorganisms and the organic raw material was top-dressed, as a result of the weed control test performed after top-dressing the organic fertilizer 100 prepared using the antagonistic, fermentative and synthetic microorganisms and the organic raw material, organic acids and bioactive substances produced by antagonistic, fermentative and synthetic microorganisms promote germination by breaking down dormancy by softening the seed coat of dormant weed seeds, and the dormancy was broken down by fermentation heat generated by fermentation of the organic fertilizer 100 regardless of the depth of a soil layer or the presence or absence of oxygen, and thus the seeds dormant for 5 to 10 years in a deep soil layer were also broken down. The broken-down weed seeds ultimately go through a lack of oxygen in the case of the deep soil and wither due to fermentation heat in the case of the top-soil.

While the present invention has been described with reference to the exemplary embodiments, it is not limited to the embodiments. It will be apparent to those of ordinary skill in the art that modifications or alternations can be implemented within a range described in claims, and these modifications or alternations are included in the scope of the accompanying claims of the present invention.

### INDUSTRIAL APPLICABILITY

After autumn harvesting, 80 kg per 220 square meters of the organic fertilizer manufactured using antagonistic, fermentative and synthetic microorganisms and an organic raw material according to the present invention 100 is evenly sprayed over soil covered with crop residues, and water was sprayed sufficiently to allow overhydration and left. In the following spring, when 80 kg per 220 square meters of the organic fertilizer 100 is evenly sprayed onto sprouting weeds and water is sprayed sufficiently to allow overhydration, the weeds are fertilized by fermentation heat generated by fermenting the organic fertilizer 100. When second crops are planted in the soil treated thereby, it is not necessary to use a separate compost, fertilizer or herbicide, the soil was improved in air permeability and water retentivity due to a soil aggregate, and therefore tillage is not needed.

In addition, when approximately 10 wt % of the organic fertilizer 100 is added to livestock feed, the digestibility in livestock is improved, a growth stress is removed, the resistance to a disease is increased, the odor of livestock excreta is reduced, and the quality and taste of livestock products are enhanced.

In addition, when approximately 10 wt % of the organic fertilizer 100 is added to fish feed, due to effects of increased digestibility caused by promotion of digestion and absorption in various types of fish (freshwater fish and sea water fish), an increased growth rate, an increased gain rate, an increased spawning rate, an increased hatching rate and reduced excreta, feed loss is prevented and feed benefits are increased. The unique quality and taste of fish become better by increasing the production of polyunsaturated fatty acids such as DHA and EPA in fish, producing a high quality protein due to the activation of cell proliferation, and maintaining breed-specific functionality due to activation of bowel and liver functions, and by the addition of the organic fertilizer 100 to fish feed, the immunity of fish increases, disease resistance increases, and the natural healing power to external wounds and the defensive power against bacterial contamination increase, and therefore there is no need to use antibiotics or growth stimulants. For this reason, the quality of water environments is stabilized, thereby reducing stresses in fish and increasing a survival rate thereof, and feed remaining in water after feeding is fermented to prevent and improve water contamination, thereby removing fishy smells and odors.

The present invention is effective not only in the agricultural, livestock and fishing industries but also in the environmental industry.

1. A method for manufacturing an organic fertilizer using antagonistic, fermentative and synthetic microorganisms and an organic raw material according to the present invention, the method comprising:
a mixing step for producing a mixture by spraying a microorganism additive solution produced by a step of culturing microorganisms by any one or more of single, mixed or combined cultivation of antagonistic, fermentative and synthetic microorganisms isolated from soil; a step of preparing a microorganism culture medium by adding any one or more of complex amino acids to primary metabolites, secondary metabolites, complex enzymes such as lyases and ligases, antibiotics, bioactive substances and inducers, which are generated in the step of culturing microorganisms; and a step of preparing a microorganism additive solution for mixing the microorganism culture medium prepared in the step of preparing a microorganism culture medium with a molasses diluent, onto a powder-type organic raw material, which is produced by a foreign material removal step for removing impurities and foreign materials of an organic raw material, a sterilization step for sterilizing the organic raw material with steam, and a crushing step for crushing the organic raw material;
a fermentation step for fermenting the mixture produced in the mixing step; and
a drying step for drying the mixture having undergone the fermentation step.

2. The method of claim 1, wherein the organic raw material in the step of removing foreign materials is prepared by mixing 74 wt % of cereal bran, 10 wt % of expeller cake, 10 wt % of roasted chaff and rice straw flour, and 6 wt % of an additive.

3. The method of claim 2, wherein the additive is prepared by mixing any one or more of grinded fish meal, grinded bone meal and grinded crab shell meal.

4. The method of claim 1, wherein the step of culturing microorganisms includes a first cultivation step for single cultivation or mixed cultivation of any one or more of the antagonistic microorganisms, the fermentative microorganisms and the synthetic microorganisms and a second cultivation step for combined cultivation of the microorganisms having undergone the first cultivation step.

5. The method of claim 4, wherein the first cultivation step consists of any one or more of commensalism, synergism and mutualism between microorganisms.

6. The method of claim 4, wherein, in the second cultivation step, any one or more of primary metabolites, secondary metabolites, complex enzymes such as lyases and ligases, antibiotics, bioactive substances and inducers is produced by exchanging genes through interspecies communication via a signal material between populations, such as a microbial pheromone.

7. The method of claim 1, wherein, in the step of culturing microorganisms, functional microorganisms exhibiting any one or more functions of soil improvement, crop growth, disease and insect pest control, biodegradation of organic contaminants, and purification of metal contaminants through gene exchange between introduced microorganisms are produced.

8. The method of claim 1, wherein, in the step of culturing microorganisms, the microorganisms are cultured up to a lag phase, an exponential phase and a stationary phase.

9. The method of claim 1, wherein the step of culturing microorganisms consists of a 9-step transfer procedure by culture tank, in which the microorganisms are sequentially cultured in the first step performed in various media, the second step performed in an Erlenmeyer flask, the third step performed in a 20 L bioreactor, the fourth step performed in a 0.5 ton culture tank, the fifth step performed in an 1 ton culture tank, the sixth step performed in a 2.5 ton culture tank, the seventh step performed in a 5 ton culture tank, the eighth step performed in a 10 ton culture tank, and the ninth step performed in a bulk culture tank.

10. The method of claim 1, wherein, in the step of preparing a microorganism additive solution, the molasses diluent is prepared by mixing 20 to 40 wt % of molasses with 60 to 80 wt % of purified water.

11. The method of claim 1, wherein, in the step of preparing a microorganism additive solution, 25 to 50 wt % of the molasses diluent is mixed with 50 to 75 wt % of the microorganism culture medium.

12. The method of claim 1, wherein, in the mixing step, the mixture is prepared to have a moisture content of 50 to 60 wt %.

13. The method of claim 1, wherein, in the fermentation step, stirring of the mixture starts at a fermentation temperature of 40° C. to 45° C., and the fermentation temperature is maintained at 50° C. or less.

14. The method of claim 1, wherein, in the fermentation step, fermentation is carried out for 3 to 10 days.

15. The method of claim 1, wherein the fermentation step consists of a first fermentation step in which a microorganism additive solution prepared using fermentative microorganisms and synthetic microorganisms is sprayed onto the organic raw material to allow fermentation for 3 to 5 days and a second fermentation step in which a microorganism additive solution prepared using antagonistic microorganisms is sprayed to allow fermentation for 3 to 5 days.

16. An organic fertilizer prepared using antagonistic, fermentative and synthetic microorganisms and an organic raw material, which is prepared by any one of the methods of claim 1.

17. The organic fertilizer of claim 16, wherein the organic fertilizer continuously provides useful substances to soil and crops by metabolisms of the antagonistic, fermentative and synthetic microorganisms loaded into the organic raw material.

18. The organic fertilizer of claim 16, wherein the organic fertilizer provides habitats and a substrate by loading the cultured antagonistic, fermentative and synthetic microorganisms into the organic raw material to protect the growth of the microorganisms.
